(19) United States
(12) Patent Application Publication
(10) Pub. No.: US 2022/0184000 A1
(43) Pub. Date: Jun. 16, 2022

(54) USE OF CANNABIDIOL IN THE TREATMENT OF DRAVET SYNDROME

(71) Applicant: GW Research Limited, Cambridge (GB)

(72) Inventors: Geoffrey GUY, Cambridge (GB); Benjamin WHALLEY, Cambridge (GB); Pabitra PATRA, Bristol (GB)

(21) Appl. No.: 17/631,069

(22) PCT Filed: Jul. 27, 2020
(86) PCT No.: PCT/GB2020/051803

§ 371 Date: Jan. 28, 2022

(30) Foreign Application Priority Data
Jul. 29, 2019 (GB) 1910803.4

(51) Int. Cl. A61K 31/05; A61P 25/08

(52) U.S. Cl. CPC A61K 31/05; A61P 25/08

(57) ABSTRACT

The present invention relates to the use of cannabidiol (CBD) for use in the treatment of disease modification in Dravet syndrome. In particular the CBD is used to improve neonatal welfare, survival and co-morbidities in patients with Dravet syndrome. Preferably the CBD used is in the form of a botanically derived purified CBD which comprises greater titan or equal to 98% (w/w) CBD and less than or equal to 2% (w/w) of other cannabinoids. The other cannabinoids present are THC at a concentration of less than or equal to 0.1% (w/w); CBD-C1 at a concentration of less titan or equal to 0.15% (w/w); CBDV at a concentration of less titan or equal to 0.8% (w/w); and CBD-C4 at a concentration of less than or equal to 0.4% (w/w). The botanically derived purified CBD preferably also comprises a mixture of both trans-THC and cis-THC. Alternatively, a synthetically produced CBD is used.

### FIELD OF THE INVENTION

The present invention relates to the use of cannabidiol (CBD) for use in the treatment of disease modification in Dravet syndrome. In particular the CBD is used to improve neonatal welfare, survival and co-morbidities in patients with Dravet syndrome.

Preferably the CBD used is in the form of a botanically derived purified CBD which comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w). The botanically derived purified CBD preferably also comprises a mixture of both trans-THC and cis-THC. Alternatively, a synthetically produced CBD is used.

### BACKGROUND TO THE INVENTION

Epilepsy occurs in approximately 1% of the population worldwide, (Thurman et al., 2011) of which 70% are able to adequately control their symptoms with the available existing anti-epileptic drugs (AED). However, 30% of this patient group, (Eadie et al., 2012), are unable to obtain seizure freedom from the AED that are available and as such are termed as suffering from intractable or “treatment-resistant epilepsy” (TRE).

Intractable or treatment-resistant epilepsy was defined in 2009 by the International League Against Epilepsy (ILAE) as “failure of adequate trials of two tolerated and appropriately chosen and used AED schedules (whether as monotherapies or in combination) to achieve sustained seizure freedom” (Kwan et al., 2009).

Individuals who develop epilepsy during the first few years of life are often difficult to treat and as such are often termed treatment resistant. Children who undergo frequent seizures in childhood are often left with neurological damage which can cause cognitive, behavioral and motor delays.

Childhood epilepsy is a relatively common neurological disorder in children and young adults with a prevalence of approximately 700 per 100,000. This is twice the number of epileptic adults per population.

When a child or young adult presents with a seizure, investigations are normally undertaken in order to investigate the cause. Childhood epilepsy can be caused by many different syndromes and genetic mutations and as such diagnosis for these children may take some time.

The main symptom of epilepsy is repeated seizures. In order to determine the type of epilepsy or the epileptic syndrome that a patient is suffering from an investigation into the type of seizures that the patient is experiencing is undertaken. Clinical observations and electroencephalography (EEG) tests are conducted and the type(s) of seizures are classified according to the ILEA classification.

Generalized seizures, where the seizure arises within and rapidly engages bilaterally distributed networks, can be split into six subtypes: tonic-clonic (grand mal) seizures; absence (petit mal) seizures; clonic seizures; tonic seizures; atonic seizures and myoclonic seizures.

Focal (partial) seizures where the seizure originates within networks limited to only one hemisphere, are also split into sub-categories. Here the seizure is characterized according to one or more features of the seizure, including aura, motor, autonomic and awareness/responsiveness. Where a seizure begins as a localized seizure and rapidly evolves to be distributed within bilateral networks this seizure is known as a bilateral convulsive seizure, which is the proposed terminology to replace secondary generalized seizures (generalized seizures that have evolved from focal seizures and are no longer remain localized).

Focal seizures where the subject's awareness/responsiveness is altered are referred to as focal seizures with impairment and focal seizures where the awareness or responsiveness of the subject is not impaired are referred to as focal seizures without impairment.

Epileptic syndromes often present with many different types of seizure and identifying the types of seizure that a patient is suffering from is important as many of the standard AED's are targeted to treat or are only effective against a given seizure type/sub-type.

One such childhood epilepsy is Dravet syndrome. Onset of Dravet syndrome almost always occurs during the first year of life with clonic and tonic-clonic seizures in previously healthy and developmentally normal infants (Dravet, 2011). Symptoms peak at about five months of age. Other seizures develop between one and four years of age such as prolonged focal dyscognitive seizures and brief absence seizures.

Dravet syndrome patients suffer both focal and generalised seizures and may also experience atypical absence seizures, myoclonic absence seizures, atonic seizures and non-convulsive status epilepticus.

Seizures progress to be frequent and treatment-resistant, meaning that the seizures do not respond well to treatment. They also tend to be prolonged, lasting more than 5 minutes. Prolonged seizures may lead to status epilepticus, which is a seizure that lasts more than 30 minutes, or seizures that occur in clusters, one after another.

Prognosis is poor and approximately 14% of children die during a seizure, because of infection, or suddenly due to uncertain causes, often because of the relentless neurological decline. Patients develop intellectual disability and life-long ongoing seizures. Intellectual impairment varies from severe in 50% patients, to moderate and mild intellectual disability each accounting for 25% of cases.

The only FDA approved treatment specifically indicated for Dravet syndrome is Epidiolex® (botanically derived purified cannabidiol). Other commonly prescribed drugs include a combination of the following anticonvulsants: clobazam, clonazepam, levetiracetam, topiramate and valproic acid.

Stiripentol is approved in Europe for the treatment of Dravet syndrome in conjunction with clobazam and valproic acid. In the US, stiripentol was granted an Orphan Designation for the treatment of Dravet syndrome in 2008; however, the drug is not FDA approved.

Potent sodium channel blockers used to treat epilepsy have been found to increase seizure frequency in patients with Dravet Syndrome and are contraindicated. The most common are phenytoin, carbamazepine, lamotrigine and rufinamide.

Management may also include a ketogenic diet, and physical and vagus nerve stimulation. In addition to anti-convulsive drugs, many patients with Dravet syndrome are treated with anti-psychotic drugs, stimulants, and drugs to treat insomnia.

Cannabidiol (CBD), a non-psychoactive derivative from the cannabis plant, has demonstrated anti-convulsant properties in several anecdotal reports, pre-clinical and clinical studies both in animal models and humans. Three randomized control trials showed efficacy of the purified pharmaceutical formulation of CBD in patients with Dravet and Lennox-Gastaut syndrome.

Based on these three trials, a botanically derived purified CBD preparation was approved by FDA in June 2018 for the treatment of seizures associated with Dravet and Lennox-Gastaut syndromes.

The US FDA Label for Epidiolex discloses the use of CBD in the treatment of Dravet Syndrome, specifically for the treatment of seizures associated with this syndrome.¹ It does not disclose nor even suggest CBD use may improve behavioural comorbidities, such as social interaction and cognition. Furthermore, it is indicated for use in patients of at least two years of age and older.

In 2019 Huestis et al. reported a review based on studies of CBD's adverse effects (AEs) or toxicity.² Again, there is no disclosure of CBD's effect on behavioural comorbidities and further, the age of patients treated range from 0.4 to 62 years.

Silvestro et al. published a review of recent literature and clinical trials that studied CBD treatment in different forms of epilepsy,³ whilst an analysis published by Laux et al. in 2019 looked at a CBD Expanded Access Program (EAP) in patients with LGS or DS.⁴ As for the above documents, there is no disclosure of CBD's effect on behavioural comorbidities in these articles, with the age of patients treated beginning from infancy onwards.

The applicant has found the use of a botanically derived purified CBD in an acute mouse model of Dravet syndrome increased survival and delayed the worsening of neonatal welfare. In a chronic mouse model of Dravet syndrome, CBD administration did not show any adverse effect on motor function and gait and was able to reduce premature mortality, improve social behaviour and memory function, and reduce anxiety-like and depressive-like behaviours.

### BRIEF SUMMARY OF THE DISCLOSURE

In accordance with a first aspect of the present invention there is provided a cannabidiol (CBD) preparation for use in the treatment of disease modification of Dravet syndrome.

Preferably the CBD preparation comprises greater than or equal to 98% (w/w) CBD and less than or equal to 2% (w/w) other cannabinoids, wherein the less than or equal to 2% (w/w) other cannabinoids comprise the cannabinoids tetrahydrocannabinol (THC); cannabidiol-C1 (CBD-C1); cannabidivarin (CBDV); and cannabidiol-C4 (CBD-C4), and wherein the THC is present as a mixture of trans-THC and cis-THC.

Preferably the disease modification of Dravet syndrome is the improvement of neonatal welfare. Alternatively, the disease modification of Dravet syndrome is extending survival. Alternatively, the disease modification of Dravet syndrome is improvement of behavioural comorbidities.

In one embodiment the behavioural comorbidity is improvement of cognition. In a further embodiment the behavioural comorbidity is improvement of social interaction.

Preferably the CBD is present is isolated from cannabis plant material. More preferably at least a portion of at least one of the cannabinoids present in the CBD preparation is isolated from cannabis plant material.

Alternatively, the CBD is present as a synthetic preparation More preferably at least a portion of at least one of the cannabinoids present in the CBD preparation is prepared synthetically.

Preferably the dose of CBD is greater than 5 mg/kg/day. More preferably the dose of CBD is 20 mg/kg/day. More preferably the dose of CBD is 25 mg/kg/day. More preferably still the dose of CBD is 50 mg/kg/day.

In accordance with a second aspect of the present invention there is provided a method of treating disease modification in a patient suffering Dravet syndrome comprising administering a cannabidiol (CBD) preparation to the subject in need thereof.

Preferably the patient is a mammal, more preferably the mammal is a human.

### BRIEF DESCRIPTION OF THE DRAWINGS

Embodiments of the invention are further described hereinafter with reference to the accompanying drawings, in which:

FIG. 1 shows the chronic administration of CBD to wild type (WT) and Scn1a^{-/-} mice on neonatal welfare (TNW) score and survival. A: Neonatal welfare score and B: Survival;

FIG. 2 shows the chronic administration of CBD to Scn1a^{+/-} mice on survival. A: Survival and B: Percentage of Scn1a^{+/-} vehicle-treated and Scn1a^{+/-} CBD-treated animals that survived until the completion of experiment (P52);

FIG. 3 shows box and whisker plots showing chronic administration of CBD to Scn1a^{+/-} mice on motor function and gait. A: Mean time (seconds) spent on accelerated rotarod; B: Median number of foot slips made in static beam test; C: Mean left stride length (mm); D: mean right stride length and E: mean stride width; and

FIG. 4 shows box and whisker plots showing the effect of chronic administration of CBD to Scn1a^{+/-} mice on active social interaction, rearing, anxiety-like and depression-like behaviours and cognition. A: Mean time (seconds) spent on active interaction in social interaction; B: Median number of rearing made in social interaction test; C: Mean time (second) spent on open arms in Elevated Plus Maze (EPM) test; D: Mean sucrose preference (%) in Sucrose Preference test; E. Mean reference memory errors (RME); and F: Median working memory errors (WME).

### DEFINITIONS

Definitions of some of the terms used to describe the invention are detailed below:

Over 100 different cannabinoids have been identified, see for example, Handbook of Cannabis, Roger Pertwee, Chapter 1, pages 3 to 15. These cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the cannabis plant. The phytocannabinoids can be isolated from plants to produce a highly purified extract or can be reproduced synthetically.

“Highly purified cannabinoids” are defined as cannabinoids that have been extracted from the cannabis plant and purified to the extent that other cannabinoids and non-cannabinoid components that are co-extracted with the cannabinoids have been removed, such that the highly purified cannabinoid is greater than or equal to 95% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

Human equivalent dose calculation****

### DETAILED DESCRIPTION

### Preparation of Botanically Derived Purified CBD

The following describes the production of the botanically derived purified CBD which comprises greater than or equal to 98% w/w CBD and less than or equal to other cannabinoids was used in the open label, expanded-access program described in Example 1 below.

In summary the drug substance used in the trials is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 95% CBD w/w, typically greater than 98% w/w.

The Cannabis sativa L. plants are grown, harvested, and processed to produce a botanical extract (intermediate) and then purified by crystallization to yield the CBD (botanically derived purified CBD).

The plant starting material is referred to as Botanical Raw Material (BRM); the botanical extract is the intermediate; and the active pharmaceutical ingredient (API) is CBD, the drug substance.

All parts of the process are controlled by specifications. The botanical raw material specification is described in Table A and the CBD API is described in Table B.

TABLE A
CBD botanical raw material specification

| Test | Method | Specification |
|---|---|---|
| Identification: | | |
| A | Visual | Complies |
| B | TLC | Corresponds to standard (for CBD & CBDA) |
| C | HPLC/UV | Positive for CBDA |
| Assay: | In-house | NLT 90% of assayed |
| CBDA + CBD | (HPLC/UV) | cannabinoids by peak area |
| Loss on Drying | Ph.Eur. | NMT 15% |
| Aflatoxin | UKAS method | NMT 4 ppb |
| Microbial: | Ph.Eur. | NMT10⁷ cfu/g |
| TVC | | NMT10⁵ cfu/g |
| Fungi | | NMT10² cfu/g |
| E. coli | | |
| Foreign Matter: | Ph.Eur. | NMT 2% |
| Residual Herbicides and | Ph.Eur. | Complies |
| Pesticides | | |

TABLE B
Specification of an exemplary botanically derived purified CBD preparation

| Test | Test Method | Limits |
|---|---|---|
| Appearance | Visual | Off-white / pale yellow crystals |
| Identification A | HPLC-UV | Retention time of major peak corresponds to |
| | | certified CBD Reference Standard |
| Identification B | GC-FID/MS | Retention time and mass spectrum of major |
| | | peak corresponds to certified CBD Reference |
| | | Standard |
| Identification C | FT-IR | Conforms to reference spectrum for certified |
| | | CBD Reference Standard |
| Identification D | Melting Point | 65-67° C. |
| Identification E | Specific Optical | Conforms with certified CBD Reference |
| | Rotation | Standard; −110° to −140° (in 95% ethanol) |
| Total Purity | Calculation | ≥98.0% |
| Chromatographic Purity 1 | HPLC-UV | ≥98.0% |
| Chromatographic Purity 2 | GC-FID/MS | ≥98.0% |
| CBDA | HPLC-UV | NMT 0.15% w/w |
| CBDV | | NMT 1.0% w/w |
| THC | | NMT 0.1% w/w |
| CBD-C4 | | NMT 0.5% w/w |
| Residual Solvents: | GC | NMT 0.5% w/w |
| Alkane | | NMT 0.5% w/w |
| Ethanol | | |
| Residual Water | Karl Fischer | NMT 1.0% w/w |

The purity of the botanically derived purified CBD preparation was greater than or equal to 98%. The botanically derived purified CBD includes THC and other cannabinoids, e.g., CBDA, CBDV, CBD-C1, and CBD-C4.

Distinct chemotypes of the Cannabis sativa L. plant have been produced to maximize the output of the specific chemical constituents, the cannabinoids. Certain chemovars produce predominantly CBD. Only the (−)-trans isomer of CBD is believed to occur naturally. During purification, the stereochemistry of CBD is not affected.

### Production of CBD Botanical Drug Substance

An overview of the steps to produce a botanical extract, the intermediate, are as follows:

a) Growing

b) Direct drying

c) Decarboxylation

d) Extraction—using liquid CO₂

e) Winterization using ethanol

f) Filtration

g) Evaporation

High CBD chemovars were grown, harvested, dried, baled and stored in a dry room until required. The botanical raw material (BRM) was finely chopped using an Apex mill fitted with a 1 mm screen. The milled BRM was stored in a freezer prior to extraction.

Decarboxylation of CBDA to CBD was carried out using heat. BRM was decarboxylated at 115° C. for 60 minutes.

Extraction was performed using liquid CO₂ to produce botanical drug substance (BDS), which was then crystalized to produce the test material. The crude CBD BDS was winterized to refine the extract under standard conditions (2 volumes of ethanol at −20° C. for approximately 50 hours). The precipitated waxes were removed by filtration and the solvent was removed to yield the BDS.

### Production of Botanically Derived Purified CBD Preparation

The manufacturing steps to produce the botanically derived purified CBD preparation from BDS were as follows:

a) Crystallization using C₅-C₁₂ straight chain or branched alkane

b) Filtration

c) Vacuum drying

The BDS produced using the methodology above was dispersed in C₅-C₁₂ straight chain or branched alkane. The mixture was manually agitated to break up any lumps and the sealed container then placed in a freezer for approximately 48 hours. The crystals were isolated via vacuum filtration, washed with aliquots of cold C₅-C₁₂ straight chain or branched alkane, and dried under a vacuum of <10 mb at a temperature of 60° C. until dry. The botanically derived purified CBD preparation was stored in a freezer at −20° C. in a pharmaceutical grade stainless steel container, with FDA food grade approved silicone seal and clamps.

### Physicochemical Properties of the Botanically Derived Purified CBD

The botanically derived purified CBD used in the clinical trial described in the invention comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w).

The botanically derived purified CBD used additionally comprises a mixture of both trans-THC and cis-THC. It was found that the ratio of the trans-THC to cis-THC is altered and can be controlled by the processing and purification process, ranging from 3.3:1 (trans-THC:cis-THC) in its unrefined decarboxylated state to 0.8:1 (trans-THC:cis-THC) when highly purified.

Furthermore, the cis-THC found in botanically derived purified CBD is present as a mixture of both the (+)-cis-THC and the (−)-cis-THC isoforms.

Clearly a CBD preparation could be produced synthetically by producing a composition with duplicate components.

Example 1 below describes the use of a botanically derived purified CBD in an acute mouse model of Dravet syndrome that CBD increased survival and delayed the worsening of neonatal welfare. In a chronic mouse model of Dravet syndrome, CBD administration did not show any adverse effect on motor function and gait and was able to reduce premature mortality, improve social behaviour and memory function, and reduce anxiety-like and depressive-like behaviours.

### Example 1: Cannabidiol (CBD) in an Acute and Chronic Mouse Model of Dravet Syndrome to Test Survival and Comorbidity

### Methods

### Study I: Assessment of Neonatal Welfare and Survival in Scn1a^{-/-} Mice

### Animals:

129S-Scn1a^{tm1Keammjax} heterozygote mice (Jackson Laboratory, USA) were maintained in and bred together to obtain Scn1a^{-/-} and wild type (WT) animals used for this Study (n=10 per group).

The maternal behaviour of the dams was also assessed simultaneously to ensure that any of the parameters observed in the study animals (Scn1a^{-/-}/WT mice) were not affected by the dam's behaviour. In this study, dam scores remained 0 throughout the study and so the responses of the pups were not considered to have been affected by variations in maternal behaviours. At the end of the study, animals were humanely killed by a Schedule 1 method (cervical dislocation).

### Experimental Design:

Following genotyping, animals were randomly divided into four groups WT vehicle-treated, WT CBD-treated, Scn1a^{-/-} vehicle-treated and Scn1a^{-/-} CBD-treated (n=10/group). They were injected subcutaneously twice daily with either CBD (100 mg/kg) or its vehicle (ethanol:Kolliphor®:0.9% saline=2:1:17) from P8 until P25 or death (whichever was earlier).

A twice daily welfare check was conducted throughout the entire duration of the study. Drug administration was conducted at 0800 h and followed by welfare checks. Conversely, afternoon welfare checks were conducted from 1600 h and followed by drug administration in order to provide the maximum possible time between doses.

### Assessment of Welfare Scores:

Welfare scoring of neonates was conducted twice daily using a blinded spreadsheet that lacked the information on the genotype of the animals and the treatment (CBD/vehicle) given to them, to ensure the experimenter remained blind to both treatment and genotype. Neonatal welfare scoring was based upon a previously validated standardised approach used widely in murine models (Langford et al., 2010; Ullman-Culleré & Foltz, 1999; Wolfensohn & Lloyd, 2007).

The parameters used for the welfare assessment were: weight, natural activity (NA; 0-3), reflex/response to touch (RT; 0-3), orbital tightening (OT; 0-2), and body condition score (BC; 1-3). A total neonatal welfare score (TNW; range 0-8) was calculated by adding together scores from NA, RT, and ST.

### Assessment of Survival:

Animal suffering was minimised by employing a validated, welfare scoring system alongside a mathematical model to predict death. In this way, any animal for which the model predicted death could be sacrificed 0.5 day before enduring the maximal severity of the disease. The model used an algorithm to predict death based on prior data obtained from untreated Scn1a^{-/-} mice (n=19) that exhibited the maximum severity of the disease and died a natural death (data not shown).

In this algorithm, the thresholds for each parameter (TNW, NA, RT, OT, BC scores) to predict death were obtained using the following procedure: (i) each parameter, measured every half day from birth for each animal, is averaged with a moving mean with a 1.5 day window; (ii) the least severe score for each parameter observed across the 19 animals over 0.5 day before their death was found; (iii) each of the 5 parameters exhibited by the animals in the study were compared to scores obtained in (ii) twice a day; (iv) if each of the 5 parameters reached their respective threshold defined in (ii) at least once since P8, the animal would undergo a Schedule 1 procedure (cervical dislocation) within 0.5 day. Additionally, surface temperature (ST) threshold was employed such that if the sum of the ST scores over the last 1.5 days, was equal to or greater than 3, the animal would be killed by cervical dislocation within 0.5 day.

### Study II: Assessment of Survival and Comorbidities in Scn1a+/− Mice

### Animals:

The animals were group housed throughout Study II except for 3 days during sucrose preference test when each animal was individually housed. This experiment was conducted in dark cycle (dim red light, 8:00-20:00 h). Male 129S-Scn1^{atm1Kea/Mmjax} heterozygote mice (Jackson Laboratory, USA) were bred with female wild type C57BL/6 mice (Charles River, UK) to obtain Scn1a^{+/-} and wild type (WT) littermate mice used in this study. At the end of the study, animals were humanely killed by cervical dislocation.

### Experimental Design:

Here, Scn1a^{+/-} were randomly divided into two groups and subcutaneously injected with either CBD (100 mg/kg twice daily; n=12) or its vehicle (ethanol:Kolliphor®:0.9% saline=2:1:17; n=29) from P8 onwards until P52 or death (whichever was earlier).

Similarly, wild type (WT) littermate mice (n=11) were injected with vehicle for the entire period of the study. Given that a significant number of deaths (˜60%) were predicted to occur between P20-P27 in vehicle-treated Scn1a^{+/-} a larger initial group size was utilised to obtain a minimum n=10 animals/group for behavioural assessment from P35 onwards.

### Assessment of Survival:

As seizure-related deaths in this model were unpredictable, animals were video monitored continuously (24 h×7 days) throughout the study and any mortality observed was cross checked with the available video footage to confirm the reason of death.

### Assessment of Motor Function:

Fine motor control in animals were assessed by the accelerated rotarod and static beam tests. Animals were habituated to the stationary rotarod for 2 min a day for 2 days. In the accelerated rotarod test each mouse was placed individually on a linearly accelerating rod (4-40 rpm over 5 minutes; LE8500, Letica Scientific Instruments, UK) and average latency to fall from the rod (maximum 300 seconds) was calculated from 3 consecutive trials (2 min interval between trials).

The static beam task was further employed to analyse balance and coordination (Sedy, Urdzikova, Jendelova, & Sykova, 2008), where the animals were required to walk along a cylindrical elevated beam (100 cm long, 0.9 cm diameter and 50 cm height from floor) and enter a dark enclosure at the beam end. The mice were habituated to the task for three consecutive days before the test day. Each day of the habituation period, the animals were placed 30, 60 and 100 cm away from the enclosure and allowed to traverse along the beam. On the test day, each mouse performed two consecutive trials (2 minutes interval between trials) with a maximum given time of 2 minutes to complete the task (the nose entering the box was taken as task completion). The test was video monitored (Sony DCR-SX21E) and blinded offline analysis was conducted (Observer XT 12, Noldus, The Netherlands) to evaluate the average number of foot slips made from two consecutive trials.

### Assessment of Gait:

Gait test was conducted to assess the cerebellar function of the animals (Patel & Hillard, 2001). In this test, the hind paws of each mouse were marked with a non-toxic ink and the mouse was allowed to walk on a white paper (50×10 cm) placed on the floor of a custom-made plexiglass tunnel (50×10×10 cm). To obtain the left and right stride length, the distance between two ipsilateral paw prints was measured, whereas stride width was calculated from the distance between a footprint and its contralateral stride length at right angle (Wecker et al., 2013). The initial and last footprints were not considered in measurements. All the animals were habituated to the test procedures and the apparatus for 2 days prior to the test. On the day of test, two trials were conducted for each animal to obtain mean stride length (left or right) and width for that animal.

### Assessment of Social Interaction:

The social interaction test was conducted in the home cage of test mouse to assess the social behaviour of the animals (Sato, Mizuguchi, & Ikeda, 2013). On test day, cage mate(s) were removed from the home cage of the test mouse and the mouse remained in isolation for 15 minutes. A novel wild type mouse of same strain, same sex and similar weight to the test mouse was then introduced to the home cage of the test mouse. Activity was video recorded (Sony DCR-SX21E) for 10 minutes and the obtained video files were blinded at the end of all experiments. Time spent in active interactions (e.g. close following, sniffing, allogrooming/social grooming and mounting) and number of rearing (lifting the front paws on the air) occasions were coded offline using Observer XT 12 (Noldus, Netherlands). Aggressive behaviours were not considered as social interactions and were not coded. In this test, a reduced social interaction is considered as autistic-like behaviour (Sato et al., 2013), while increased rearing occasions is sign of defensive escapes (Kaplan et al., 2017).

### Assessment of Anxiety-Like Behaviours:

The elevated plus maze (EPM) test was performed to assess the level of anxiety in animals (M. Chen et al., 2017). The wooden test apparatus consists of two closed arms (50×10×40 cm) and two open arms (50×10 cm) connected via a central platform (10×10 cm) and raised at a height of 50 cm above the floor. Each animal was placed on the central platform facing towards an open arm. Activity was video recorded (Swann SRDVR-16440H, UK) for 5 minutes. The video files were blinded and coded offline at the end of all experiments using Observer XT 12 (Noldus, Netherlands). Time spent on open arms was inversely related to the level of anxiety.

### Assessment of Depression-Like Behaviour:

The sucrose preference test was carried out to assess the depression-like behaviour (Serova, Mulhall, & Sabban, 2017). The animals were separately housed during this test. Here, 24 hours before the test, animals were trained to drink from two bottles each containing 2% sucrose. On the first day of test, the animals were provided with a pre-weighed bottle of 2% sucrose and another containing a pre-weighed volume of tap water. The positions of the bottles were swapped after 24 hours to avoid any side preferences. After 48 hours, both bottles were weighed, and sucrose preference was calculated by using the following formula:

Sucrose preference (%)=Sucrose consumption/Sucrose consumption+Water consumption×100

### Assessment of Cognition:

A radial arm maze (RAM) consisting of eight arms (each arm 60×10 cm; raised at 50 cm above the floor) was used to assess the reference memory (RM) and working memory (WM) of the animals. On four consecutive days, animals were given two 10-minute sessions of habituation to the test apparatus and rules of the test, separated by a 90 min interval. During the first two days of habituation, food rewards (¼ Cheerios®, Nestle) were randomly scattered on the floor of the apparatus covering all arms and food-troughs at the end of each arm. On the 3rd and 4th habituation day, food rewards were placed only in food troughs of four randomly selected arms (fixed for each animal during the habituation and test day). Food was withdrawn 4-6 hours before the trial (both during habituation and test days) to motivate the animals to locate the rewards and thus perform the task. On the test day two trials of 10 min were conducted at 90 min interval and the activity of the animals were video recorded for offline blinded coding after the end of experiment. Entry to a non-baited arm was considered as a reference memory error (RME), whereas re-entry to a previously baited arm from which the food was already taken is considered as a working memory error (WME). The mean WME or RME were calculated from the two test trials.

### Statistical Analysis:

The data and statistical analysis comply with the recommendations on experimental design and analysis in pharmacology (Curtis et al., 2018).

In Study I, the welfare parameters were analysed using SPSS 24 (IBM SPSS Statistics®, UK), whilst survival data were analysed using GraphPad Prism 6 software (GraphPad Software, Inc., USA). Data obtained from welfare parameters were compared using a three-way ANOVA to observe the main effects of treatment, genotype and time, and their two-way and three-way interactions. If significant two-way interactions were found Bonferroni post hoc tests were conducted on any treatment×genotype interactions to assess the effect of CBD treatment on different genotypes (WT/Scn1a^{-/-}). Bonferroni post hoc tests were also conducted for any significant three-way treatment×genotype×time interactions to compare the effect of CBD treatment with vehicle treatment at every time point of welfare assessment in both the WT and Scn1a^{-/-} groups. In all cases, post hoc analyses were corrected for multiple comparisons. Data from 2.2% welfare scores were outliers and were excluded from further analysis (±2.5*SD) (J. Miller, 1991). For the survival data, survival curves from Scn1a^{-/-} vehicle-treated and CBD-treated groups were compared using a Mantel-Cox test. No WT animals died during the study, so survival curves were not compared. All the data are expressed as mean±SEM. In all cases, p<0.05 is considered as the level of significance.

In Study II, the data were analysed in GraphPad Prism 6 software. Survival curves of Scn1a^{+/-} vehicle-treated and Scn1a^{+/-} CBD-treated group were compared using a Mantel-Cox test. The percentage of animals from the Scn1a^{+/-} vehicle-treated and Scn1a^{+/-} CBD-treated groups that survived until the end of the study (P52) were compared by Fisher's exact test. Further, data obtained from the comorbidity assessment were checked for normality by D'Agostino & Pearson omnibus normality test. Data obtained from rotarod, gait, social interaction (active interaction), EPM, sucrose preference, RAM (RME) tests were normally distributed and the differences between the three groups were analysed by one-way ANOVA. If a significant difference was found then Holm-Sidak post-hoc test was conducted among the groups. On the other hand, data obtained from static beam, social interaction (rearing occasions), RAM (WME) were found to be non-parametric, thus were analysed by Kruskal-Wallis test. Upon observing a significant difference, the Dunn's post-hoc test was employed to compare the groups. Multiple comparisons were corrected in all cases. Parametric data are presented in scattered dot plot in the figures and are expressed as mean±SEM. Non-parametric data are presented in box plot in the figures and are expressed as median, min to max, and interquartile range (IQR). In all cases, p<0.05 was considered to be the level of significance.

### Results

### Study I: Neonatal Welfare in Scn1a^{-/-} Mice

In this Study, animals (n=10/group) were treated with either vehicle or CBD from P8 until P25 or death (whichever was earlier), and welfare was monitored twice daily. The mean total neonatal welfare (TNW) scores in WT vehicle-treated, WT CBD-treated, Scn1a^{-/-} vehicle-treated and Scn1a^{-/-} CBD-treated group were respectively 0.39±0.04, 0.24±0.04, 3.66±0.04 and 2.85±0.04. Main effects of treatment (F(1,612)=128.78; p<0.001), genotype (F(1,612)=4850.12; p<0.001) and time (F(16,612)=57.89; p<0.001) on TNW scores was found.

A significant three-way interaction among treatment×genotype×time was observed (F(16,612)=5.46, p<0.001), whilst significant two-way interactions were observed for treatment×genotype (F(1,612)=62.74; p<0.001), treatment×time (F(16,612)=2.19; p=0.005) and genotype×time (F(16,612)=112.22; p<0.001).

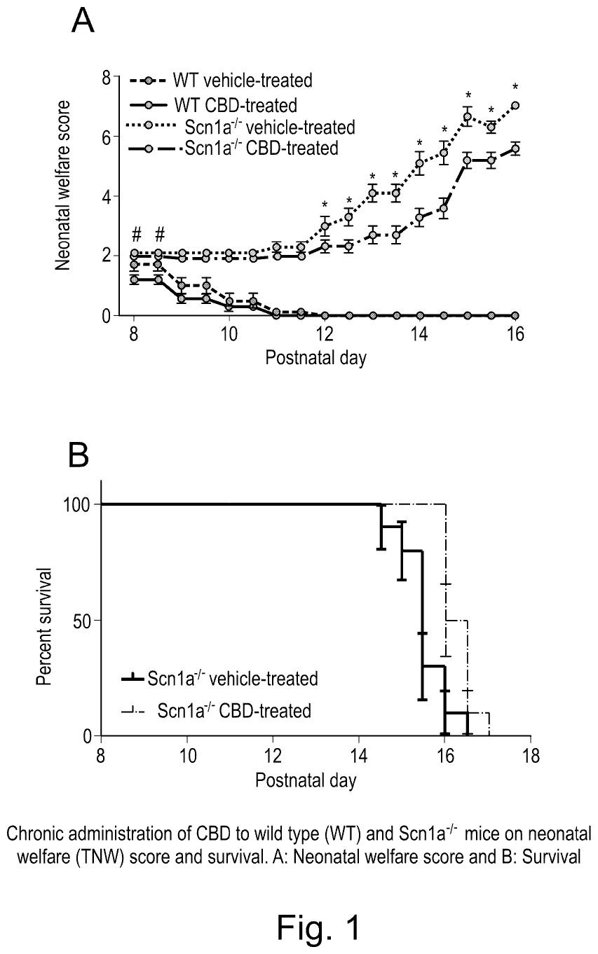

The post hoc comparison for treatment×genotype×time interactions revealed that CBD delayed the worsening of welfare scores in Scn1a^{-/-} mice from P12 to P16 compared to the vehicle-treated Scn1a^{-/-} mice on respective days (p<0.01; FIG. 1A). This post hoc test further showed that CBD improved TNW score in WT animals from P8-P8.5 i.e. in first day of treatment compared to the WT vehicle-treated animals on respective occasions (p<0.05; FIG. 1A).

### Study I: Survival in Scn1a^{-/-} Mice

None of the WT animals died during the study. In the two Scn1a^{-/-} groups the median survival in the CBD-treated Scn1a^{-/-} mice was significantly higher (16.25 days) compared to the vehicle-treated Scn1a^{-/-} mice (15.5 days; X2=8.61; p=0.003; n=10/group; FIG. 1B).

### Study II: Premature Mortality in Scn1a^{+/-} Mice

The mortality rate was highest between P20-P27 in Scn1a^{+/-} mice except for a single animal from the Scn1a^{+/-} vehicle-treated group which died at P47. The recorded video footage was reviewed, and it was confirmed that tonic-clonic seizures were the cause of death in all cases.

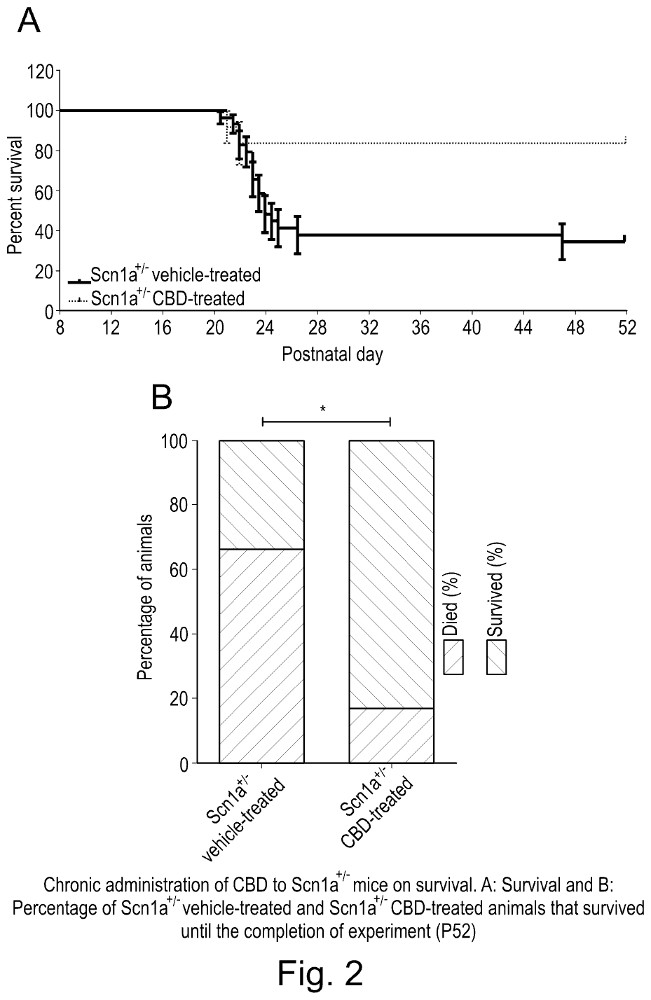

Survival was significantly less in Scn1a^{+/-} vehicle-treated group compared to the Scn1a^{+/-} CBD-treated group (X2=5.94; p=0.04; FIG. 2A). Approximately 66% (19 of 29) Scn1a^{+/-} vehicle-treated animals died before the completion of the study in contrast to only 17% (2 of 12) Scn1a^{+/-} CBD-treated animals (p<0.0001; FIG. 2B).

### Study II: Effect on Motor Function

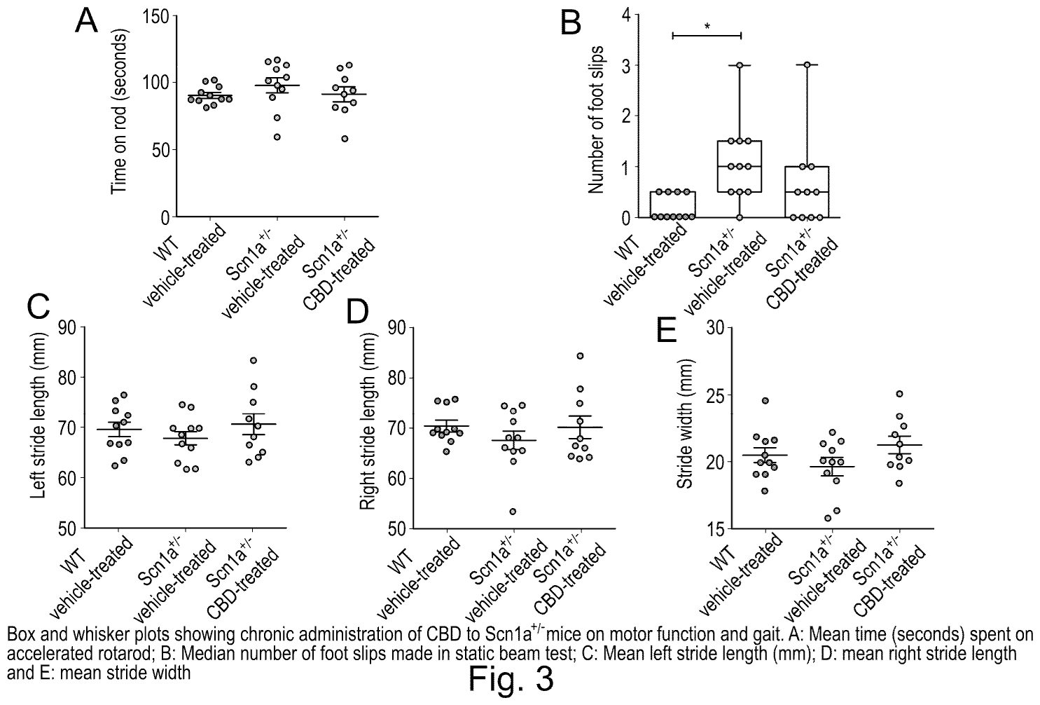

Motor function was assessed by both the accelerating rotarod and static beam test. In the accelerating rotarod test, no significant difference in time spent on rod was observed between the groups (F(2,29)=0.86; p=0.44; FIG. 3A).

In the static beam test, a significant difference in number of foot slips was found among the groups (H(2)=10.67; p=0.005). Scn1a^{+/-} vehicle-treated group made significantly (p=0.003) more foot slips compared to the WT vehicle-treated group, however no significant difference was observed in between Scn1a^{+/-} vehicle-treated and Scn1a^{+/-} CBD-treated groups (p=0.23; FIG. 3B). A further comparison between the WT vehicle-treated and the Scn1a^{+/-} CBD-treated groups revealed no significant difference in number of foot slips between these two groups (p=0.48).

### Study II: Gait Abnormalities

In the gait test, no significant change was observed for left stride length (F(2,29)=0.73; p=0.44; FIG. 3C), right stride length (F(2,29)=0.86; p=0.44; FIG. 3D) and stride width (F(2,29)=1.87; p=0.17; FIG. 3E) between the three groups.

### Study II: Social Behaviour of Scn1a^{+/-} Mice

The social interaction test was conducted to assess the active social interaction and rearing behaviour exhibited in the home cage of the test animals.

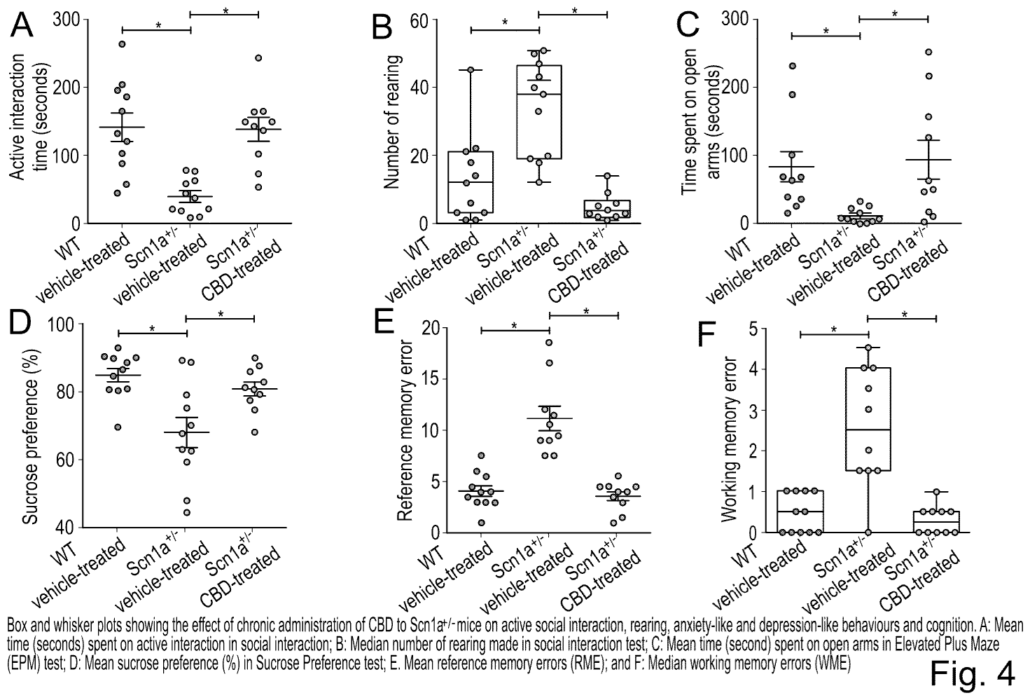

The time spent on active interaction was significantly differed among the groups (F(2,29)=13.58; p<0.0001). The Scn1a^{+/-} vehicle-treated animals (n=11) spent significantly less time in performing active interaction with the stranger mouse compared to both WT vehicle-treated (n=11; p=0.0002) and Scn1a^{+/-} CBD-treated (n=10) animals (p=0.0003; FIG. 4A). The active interaction by the Scn1a^{+/-} CBD-treated group was similar to the WT vehicle-treated group (p=0.86).

On the other hand, a significant difference in number of rearing events was observed among the groups (H(2)=16.18; p=0.0003) with a significantly higher number of rearing occasions for Scn1a^{+/-} vehicle-treated animals compared to both WT vehicle-treated (p=0.02) or Scn1a^{+/-} CBD-treated (p=0.0003) animals (FIG. 4B). No difference in rearing events was observed between the WT vehicle-treated and Scn1a^{+/-} CBD-treated groups (p=0.55).

### Study II: Anxiety-Like Behaviour in Scn1a^{+/-} Mice

The anxiety of the animals was assessed by the amount of the time spent on the open arms of an EPM. The time spent on the open arms differs significantly among the groups (F(2,28)=5.11; p=0.01). The Scn1a^{+/-} vehicle-treated animals (n=11; FIG. 4C) spent significantly less time on the open arms compared to both WT vehicle-treated (n=11; p=0.03) and Scn1a^{+/-} CBD-treated (n=10) animals (p=0.02). The time spent on the open arms was not different between WT vehicle-treated and Scn1a^{+/-} CBD-treated groups (p=0.73).

### Study II: Depression-Like Behaviour in Scn1a+/− Mice

Depression-like behaviour is inversely correlated with sucrose preference (Murray, Boss-Williams, & Weiss, 2013). Sucrose preference differed significantly among the groups (F(2,29)=8.37; p=0.001). Scn1a^{+/-} vehicle-treated animals (n=11; FIG. 4D) had a reduced preference for sucrose in comparison to both WT vehicle-treated (n=11; p=0.002) or Scn1a^{+/-} CBD-treated (n=10; p=0.01) animals. Sucrose preference was similar in between the WT vehicle-treated and Scn1a^{+/-} CBD-treated groups (p=0.36).

### Study II: Cognition in Scn1a^{+/-} Mice

The reference memory and working memory function in the animals were assessed using an eight-arm RAM test. A significant difference (F(2,28)=29.54; p<0.0001) in the number of RME was observed among the groups. The Scn1a^{+/-} vehicle-treated group (n=10) made significantly more RME compared to both WT vehicle-treated (n=11) and Scn1a^{+/-} CBD-treated (n=10) groups (p<0.0001; FIG. 4E). No difference in RME was observed between the WT vehicle-treated and Scn1a^{+/-} CBD-treated groups (p=0.65).

Further, WME were significantly different among the groups (H(2)=15.22; p=0.0005). The Scn1a^{+/-} vehicle-treated group made significantly more WME compared to both WT vehicle-treated (p=0.004) and Scn1a^{+/-} CBD-treated groups (p=0.001; FIG. 4F). The WME was not differed in Scn1a^{+/-} CBD-treated group compared to the WT vehicle-treated group (p>0.9999).

### CONCLUSIONS

These data indicate that CBD was able to improve neonatal welfare and extend survival in an acute model of Dravet syndrome using Scn1a^{-/-} mice.

Additionally, it was found that chronic administration of CBD was able to prevent premature mortality and improve several behavioural comorbidities, including impaired cognition and social interaction, in a chronic model of Dravet syndrome in Scn1a^{+/-} mice.

Such data are indicative of a disease modifying effect of CBD in the treatment of Dravet syndrome.

CBD produced no detrimental effects on motor function which are often found with the current pharmacotherapy for this disorder.

### REFERENCES

1. US FDA Epidiolex label (2018) https://www.accessdata.fda.gov/drugsatfda_docs/label/2018/210365lbl.pdf
2. Huestis et al. (2019) “Cannabidiol Adverse Effects and Toxicity.” Current Neuropharmacology, 17(10):974-989 https://europepmc.org/article/med/31161980
3. Silvestro et al. (2019) “Use of Cannabidiol in the Treatment of Epilepsy: Efficacy and Security in Clinical Trials.” Molecules 2019, 24(8), 1459 https://www.mdpi.com/1420-3049/24/8/1459/htm
4. Laux et al. (2019) “Long-term safety and efficacy of cannabidiol in children and adults with treatment resistant Lennox-Gastaut syndrome or Dravet syndrome: Expanded access program results” Epilepsy Research Volume 154, August 2019, Pages 13-20 https://www.sciencedirect.com/science/article/pii/S0920121118305837?via%3Dihub

1. A method of treating disease modification in a patient suffering Dravet syndrome comprising administering a cannabidiol (CBD) preparation to the subject in need thereof.

2. The method according to claim 1, wherein the CBD preparation comprises greater than or equal to 98% (w/w) CBD and less than or equal to 2% (w/w) other cannabinoids, wherein the less than or equal to 2% (w/w) other cannabinoids comprise the cannabinoids tetrahydrocannabinol (THC); cannabidiol-C1 (CBD-C1); cannabidivarin (CBDV); and cannabidiol-C4 (CBD-C4), and wherein the THC is present as a mixture of trans-THC and cis-THC.

3. The method according to claim 1, wherein the disease modification of Dravet syndrome is the improvement of neonatal welfare.

4. The method according to claim 1, wherein the disease modification of Dravet syndrome is extending survival.

5. The method according to claim 1, wherein the disease modification of Dravet syndrome is improvement of behavioral comorbidities.

6. The method according to claim 5, wherein the behavioral comorbidity is improvement of cognition.

7. The method according to claim 5, wherein the behavioral comorbidity is improvement of social interaction.

8. The method according to claim 1, wherein the CBD is isolated from cannabis plant material.

9. The method according to claim 1, wherein at least a portion of at least one of the cannabinoids present in the CBD preparation is isolated from cannabis plant material.

10. The method according to claim 1, wherein the CBD is present as a synthetic preparation.

11. The method according to claim 10, wherein at least a portion of at least one of the cannabinoids present in the CBD preparation is prepared synthetically.

12. The method according to claim 1, wherein the dose of CBD is greater than 5 mg/kg/day.

13. The method according to claim 1, wherein the dose of CBD is 20 mg/kg/day.

14. The method according to claim 1, wherein the dose of CBD is 25 mg/kg/day.

15. The method according to claim 1, wherein the dose of CBD is 50 mg/kg/day.

16. (canceled)
